(19) United States
(12) Patent Application Publication
(10) Pub. No.: US 2025/0144473 A1
(43) Pub. Date: May 8, 2025

(54) SYSTEM AND METHOD FOR DETERMINING IMPACT ON BODY PART AND RECOMMENDING EXERCISE

(71) Applicant: Samsung Electronics Co., Ltd., Suwon-si (KR)

(72) Inventor: Vijayanand KUMAR, Noida (IN)

(73) Assignee: SAMSUNG ELECTRONICS CO., LTD., Suwon-si (KR)

(21) Appl. No.: 19/002,227

(22) Filed: Dec. 26, 2024

Related U.S. Application Data
(63) Continuation of application No. PCT/KR2023/012488, filed on Aug. 23, 2023.

(30) Foreign Application Priority Data
Aug. 26, 2022 (IN) 202211048874

(51) Int. Cl. A63B 24/00; A61B 5/00; A61B 5/11; A63B 71/06; G06F 3/0346; G06F 3/0484; G06F 3/0488

(52) U.S. Cl. CPC A63B 24/0075; A61B 5/1116; A61B 5/486; A61B 5/7264; A63B 71/0622; G06F 3/0346; A61B 2562/0219; G06F 3/0484; G06F 3/0488

(57) ABSTRACT

A method may include: receiving inertial sensor data and touch screen data of the user equipment (UE); determining an application type running on the UE; predicting, by a neural network, a holding orientation of the UE based on the inertial sensor data, the application type, and the touch screen data; determining, by the neural network, a body posture of the user and at least one impacted body part based on the inertial sensor data, based on the holding orientation; determining, by the neural network, an impact level of the at least one impacted body part based on the body posture, the holding orientation of the UE and the inertial sensor data of the UE; and recommending a body posture correction and the at least one exercise for the at least one impacted body part based on the impact level.

### CROSS-REFERENCE TO RELATED APPLICATIONS

This application is a continuation application of International Application No. PCT/KR2023/012488 designating the United States, filed on Aug. 23, 2023, in the Korean Intellectual Property Receiving Office and claiming priority to Indian Patent Application number 202211048874, filed on Aug. 26, 2022, in the Indian Patent Office, the disclosures of which are incorporated by reference herein in their entireties.

### BACKGROUND

### 1. Field

The disclosure generally relates to recommending exercise, and more particularly, to systems and methods for determining impact on at body part while using a user equipment (UE) and recommending exercise.

### 2. Description of Related Art

Electronic devices have become a central element in human lives. Every day-to-day activities are surrounded towards electronic devices and are done with their usage. Particularly, a mobile phone has become an all-time partner for a user. The users are constantly engaged with the mobile phone and often neglect their health while using the mobile phone.

The over usage of the mobile phone and an incorrect posture of a body of the user while using the mobile phone may lead to several health hazards such as text neck, etc. The incorrect posture of the body such as bended posture leading to bend in neck, head, spine may affect body parts in terms of pain, vital fluctuations, mental and overall physical problems.

FIG. 1 illustrates scenario of the body posture while using a user equipment, such as the mobile phone, according to the related art. As depicted, the user may bend the neck, spine while holding and using the mobile phone. Depending upon the angle at which the mobile phone is held, the user may bend his body posture accordingly and thus may risk subjecting body to undue physical stress due to incorrect posture. Furthermore, longer usage duration of mobile phones with bending body parts causing incorrect body postures may lead to acute or chronic pain in different body parts, affect metabolic activity, increase heart attack chances, and other severe physical and mental diseases etc.

Currently, there are a few mechanisms available for correcting the body posture of the user and recommending exercise to eliminate physical stress. However, most of the existing techniques lack real-time, hassle-free analysis of the body posture and recommending exercises to eliminate the physical stress. The existing techniques disclose usage of multiple wearables to capture the body posture, thus making the technique quite full of hassle for the user. Alternatively, the existing techniques suggest capturing body posture with the camera. However, the camera of the mobile phone may not be active at every instance and fails to continuously monitor the body posture.

Accordingly, there is a need for a methodology which may determine impact on the body part while using the mobile phone and recommend exercise to remove any physical stress.

### SUMMARY

This summary is provided to introduce a selection of concepts, in a simplified format, that are further described in the detailed description. This summary is neither intended to identify key or essential inventive concepts of the disclosure and nor is it intended for determining the scope of the disclosure.

According to an aspect of the disclosure, a method for determining impact on at least one body part while using a user equipment (UE) and recommending at least one exercise, may include: receiving inertial sensor data and touch screen data of the user equipment (UE); determining an application type running on the UE; predicting, by a neural network, a holding orientation of the UE based on the inertial sensor data, the application type, and the touch screen data, wherein the holding orientation of the UE indicates whether a user is holding and currently operating the UE; determining, by the neural network, a body posture of the user and at least one impacted body part based on the inertial sensor data, based on the holding orientation; determining, by the neural network, an impact level of the at least one impacted body part based on the body posture, the holding orientation of the UE and the inertial sensor data of the UE; and recommending a body posture correction and the at least one exercise for the at least one impacted body part based on the impact level.

According to an aspect of the disclosure, a system for determining impact on at least one body part while using a user equipment (UE) and recommending at least one exercise, the system may include: memory storing instructions; and at least one processor configured to execute the instructions, wherein the instructions, when executed by the at least one processor, cause the system: receive inertial sensor data and touch screen data of the UE; determine an application type running on the UE; predict, by a neural network, a holding orientation of the UE based on the inertial sensor data, the touch screen data, and the application type, wherein the holding orientation of the UE indicates whether a user is holding and currently operating the UE; determine, by the neural network, a body posture of the user and at least one impacted body part based on the inertial sensor data, based on the holding orientation; determine, by the neural network, an impact level of the at least one impacted body part based on the body posture, the holding orientation of the UE and the inertial sensor data of the UE; and recommend a body posture correction and the at least one exercise for the at least one impacted body part based on the impact level.

According to an aspect of the disclosure, a user equipment (UE) may include: an inertial sensor; a touch screen; at least one processor; and memory storing instructions that, when executed by the at least one processor, cause the at least one processor to: receive inertial sensor data from the inertial sensor and touch screen data from the touch screen; predict, by a neural network, a holding orientation of the UE based on the inertial sensor data and the touch screen data; determine, by the neural network, a body posture of a user and a first body part and a second body part impacted by the body posture, based on the holding orientation; determine, by the neural network, a first impact level of the first body part and a second impact level of the second body part based on the body posture; and recommend a body posture correction and at least one exercise for at least one of the first body part or the second body part, based on the first impact level and the second impact level.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 illustrates an exemplary scenario of the body posture while using a user equipment (UE), according to the related art;

FIG. 2 illustrates a schematic block diagram depicting an environment for implementation of the disclosure, according to one or more embodiments of the disclosure;

FIG. 3A illustrates an exemplary process flow comprising a method for determining impact on a body part while using the UE and recommending an exercise, according to one or more embodiments of the disclosure;

FIG. 3B illustrates an exemplary process flow comprising a method for determining impact on a body part while using the UE and recommending an exercise, according to one or more embodiments of the disclosure;

FIG. 4A illustrates an exemplary process flow comprising a method for predicting a holding orientation of the UE, according to one or more embodiments of the disclosure;

FIG. 4B illustrates an exemplary process flow comprising a method for predicting a holding orientation of the UE, according to one or more embodiments of the disclosure;

FIG. 5 illustrates an exemplary process flow comprising a method for determining a body posture, according to one or more embodiments of the disclosure;

FIG. 6 illustrates an exemplary predefined table for determining an impacted body part, according to one or more embodiments of the disclosure;

FIG. 7A illustrates an exemplary process flow comprising a method for determining an impact level of the impacted body part, according to one or more embodiments of the disclosure;

FIG. 7B illustrates an exemplary process flow comprising a method for determining an impact level of the impacted body part, according to one or more embodiments of the disclosure;

FIG. 8A illustrates an exemplary use case depicting a posture record providing the body posture and the impacted body part, according to one or more embodiments of the disclosure;

FIG. 8B illustrates an exemplary use case depicting an exemplary use case depicting a predefined posture record table providing the predefined postures according to one or more embodiments of the disclosure;

FIG. 9A illustrates another exemplary use case depicting the impacted body part and an impact level, according to one or more embodiments of the disclosure;

FIG. 9B illustrates another exemplary use case depicting the impacted body part and a type of exercise, according to one or more embodiments of the disclosure;

FIG. 10 illustrates an exemplary process flow comprising a method for receiving feedback from the user and modifying the recommended exercises, according to one or more embodiments of the disclosure;

FIG. 11 illustrates a system architecture in accordance with one or more embodiments of the present disclosure; and

FIG. 12 illustrates another system architecture, in accordance with one or more embodiments of the present disclosure.

### DETAILED DESCRIPTION

For the purpose of promoting an understanding of the principles of the disclosure, reference will now be made to the various embodiments and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are explanatory of the disclosure and are not intended to be restrictive thereof.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the disclosure. Furthermore, in terms of the construction of the device, one or more components of the device may have been represented in the drawings by conventional symbols, and the drawings may show only those specific details that are pertinent to understanding the embodiments of the disclosure so as not to obscure the drawings with details that will be readily apparent to those of ordinary skill in the art having the benefit of the description herein.

Reference throughout this specification to “an aspect”, “another aspect” or similar language means that a particular feature, structure, or characteristic described in connection with the embodiment is included in at least one embodiment of the disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

The disclosure is directed towards a method and system for determining impact on a body part while using a user equipment (UE) and recommending an exercise for the impacted body part. In an examples of the UE may be a laptop, a mobile phone, a PDA (Personal Digital Assistant), a smart phone, a multimedia device, a wearable device, etc. More specifically, the disclosure provides for mechanisms to determine the incorrect body posture and determine the impacted body parts (e.g. a first body part and a second body part) due to the incorrect body posture and recommend exercise to correct body posture and the impact on the body parts, while the user is using the UE.

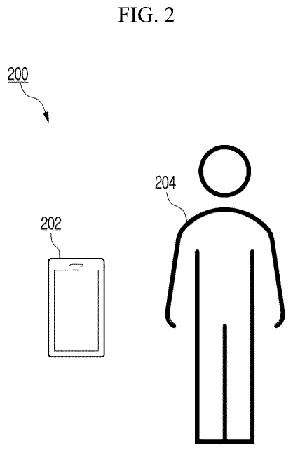

FIG. 2 illustrates a schematic block diagram depicting an environment for implementation of the disclosure, according to one or more embodiments of the disclosure.

In some embodiments, the disclosure is implemented between the UE 202, such as, but not limited to, a laptop computer, a desktop computer, a Personal Computer (PC), a notebook, a smartphone, a tablet, a smart watch, e-book readers, and a user 204 holding and operating the UE 202.

In various embodiments of the disclosure, the UE 202 is configured to acquire an inertial sensor data, a touch screen panel data, and determine an application type running on the UE 202. In an example, the inertial sensor data may be collected via an accelerometer, a gyroscope, installed in the UE 202. The accelerometer, the gyroscope may provide an angle of usage of the UE 202, a duration of usage of the UE 202, a proximity of the UE 202 to face of the user 204. In an example, the touch screen panel data may be collected via touch coordinates, hover distribution as the user 204 interacts with a touch display of the UE 202. In another example, the UE 202 may determine whether the type of application running on UE 202 such as, a video, a game, a call, a chat, etc.

In some embodiments, the UE 202 is configured to display an impacted body part of the user due to incorrect posture of the user 204 while the user 204 is holding and operating the UE 202. The UE 202 may display an exercise repetition distribution which includes a frequency and a type of the exercise along with a video, to be performed by the user 204 for the impacted body part.

In some embodiments, the UE 202 is configured to receive feedback from the user 204 via the touch display of the UE 202. In an example, the feedback may include if the user 204 performed the recommend exercise or not and a level relief achieved by the user 204 after performing the recommend exercise. The UE 202 may accordingly, update the exercise repetition distribution based on the feedback.

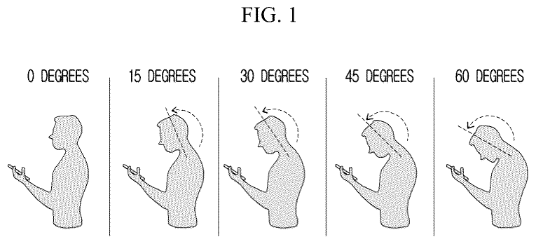

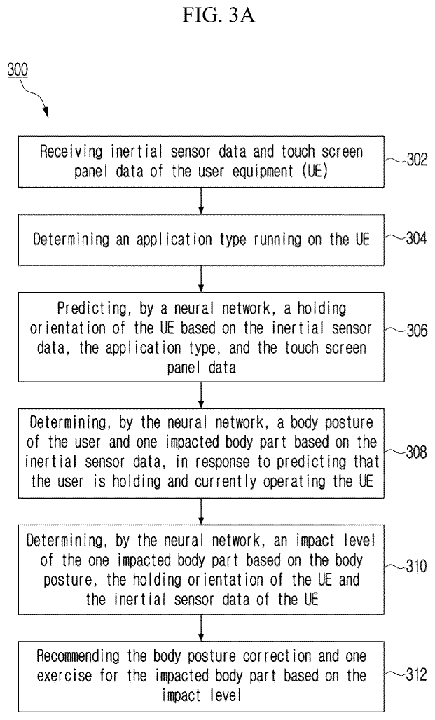

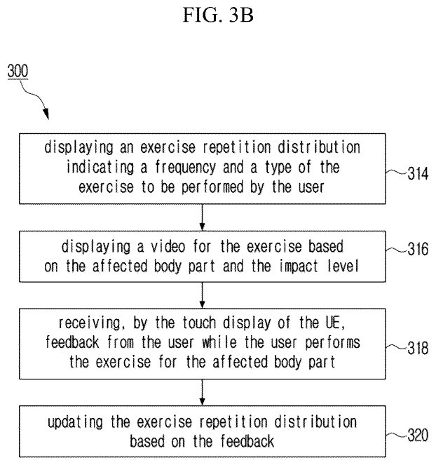

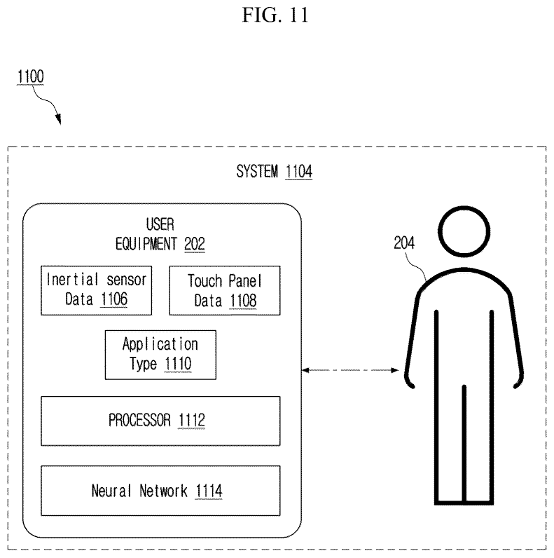

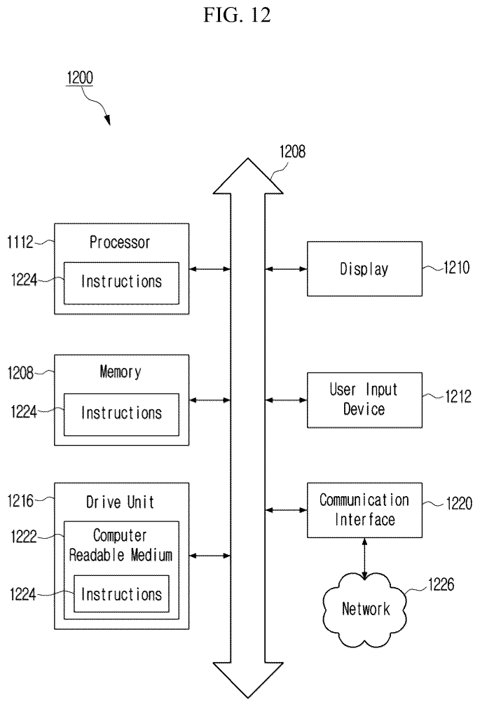

FIG. 3A and FIG. 3B illustrate an exemplary process flow comprising a method 300 for determining impact on the body part while using the UE 202 and recommending the exercise, according to one or more embodiments of the disclosure. The method 300 may be a computer-implemented method executed, for example, by the UE 202. For the sake of brevity, constructional and operational features are explained in the description of FIG. 1, FIG. 2, FIG. 3A, FIG. 3B, FIG. 11, and FIG. 12

At step 302, the method may 300 include the UE 202 receiving the inertial sensor data and the touch screen panel data. The inertial sensor data may be received from the gyroscope, the accelerometer installed in the UE 202. The touch screen panel data may be received as a result of the user 204 interacting with the touch display of the UE 202.

At step 304, the method may 300 include the UE 202 determining the type of application running on the UE 202. In an example, the application may be a video, a song, a game or a call.

At step 306, the method 300 may include predicting, by a neural network, a holding orientation of the UE 202. In an example, the holding orientation of the UE 202 is representative of whether the user 204 is holding and currently operating the UE 202. In one or more embodiments, the neural network is a fully connected artificial neural network and is trained to predict that the user 204 is clamping the UE 202 and is not actively engaged in operating the UE 202. In another embodiment, the neural network is trained to predict that the user 204 is holding and currently operating the UE 202. In the method 300, the neural network is configured to predict the holding orientation based on the inertial sensor data, the application type, and the touch screen panel data.

At step 308, the method 300 may include determining, by the neural network, the body posture of the user 204 and one impacted body part. In an example, as the user 204 is holding and currently operating the UE 202, the body posture of the user 204 is predicted based on the inertial sensor data. In the example, corresponding to the body posture of the user 204, the neural network provides the body part which may be impacted due to the body posture of the user 204 while holding and currently operating the UE 202.

At step 310, the method 300 may include the neural network determining an impact level of the impacted body part based on the body posture, the holding orientation of the UE 202 and the inertial sensor data of the UE 202. In an example, the impact level may include a level of impact on the impacted body part such as High, Medium, Low for each of the impacted body part of the user 204.

At step 312, the method 300 may include the neural network recommending the body posture correction and the exercise for the impacted body part based on the impact level.

At step 314, the method 300 may include displaying on the UE 202, an exercise repetition distribution. In an example, the exercise repetition distribution may be representative of a frequency and a type of the recommended exercise to be performed by the user 204 for correcting the body posture. In an example, the exercise repetition distribution is based on the impact level and is displayed for each of the impacted body part.

At step 316, the method 300 may include displaying on the UE 202, a video of the recommended exercise based on the affected body part and the impact level. In an example, the video may be prestored on a cloud server. In the example, thus based on the recommended exercise, the UE 202 may be configured to fetch the video stored corresponding to the recommended exercise from the cloud server and display it to the user 204.

At step 318, the method 300 may include the UE 202 receiving feedback from the user 204 via the touch display of the UE 202. In one or more embodiments, the feedback represents the level of relief in the posture correction the user 204 has achieved post performing the recommended exercise.

At step 320, the method 300 may include the UE 202 updating the exercise repetition distribution based on the feedback received from the user 204.

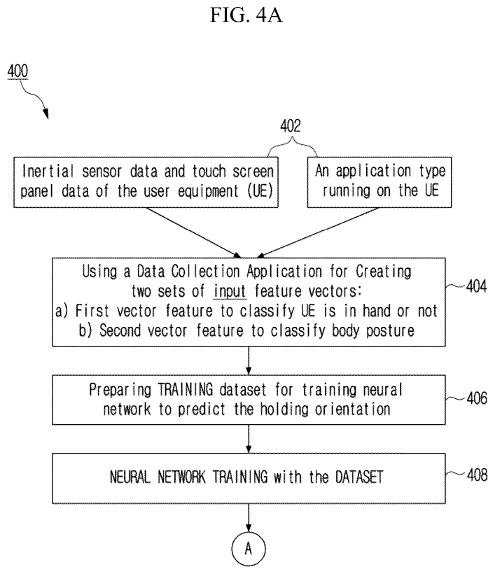

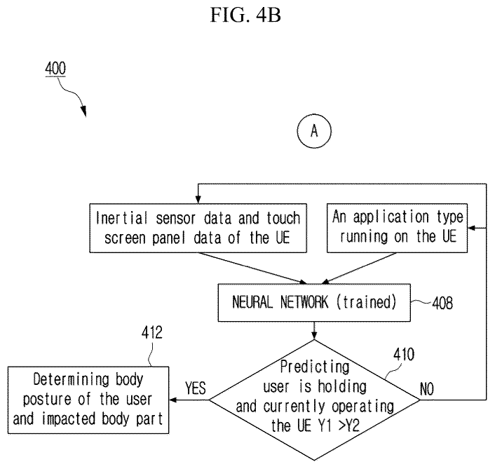

FIG. 4A illustrates an exemplary process flow comprising a method 400 for predicting the holding orientation of the UE 202, according to one or more embodiments of the disclosure.

As previously discussed with reference to FIG. 3A, at step 402, the method 400 may include receiving the inertial sensor data, the touch input data and the type of application running on the UE 202 for a N-second. In an example, the N-second may include minimum 1-second and maximum 5-seconds data.

At step 404, the method 400 may include a data collection application installed in the UE 202 and is adapted to create two set of input feature. In one or more embodiments, the data collection application may be adapted to capture all types of possible scenario indicating if the user is holding or clamping the UE 202. In one or more embodiments, a first vector feature may be created from the inertial sensor data, the touch screen panel data and the type of application collected for the N-second. The neural network may be adapted to process the first vector feature to determine if the user 204 is holding and currently operating the UE 202. In another embodiment, a second vector feature may be created from the inertial sensor data. The neural network may be adapted to process the second vector feature to determine the body posture of the user 204.

At step 406, the method 400 may include preparing a training dataset for training the neural network. In an example, the training dataset is prepared using the first vector feature. In one or more embodiments, a label is provided to each data of the prepared first vector feature. The label may indicate if the feature represents the user 204 holding the UE 202 or clamping the UE 202. In an example, the label with holding the UE 202 may represent that the UE 202 may be in-hand of the user 204 and the user 204 may be currently operating the UE 202. It may include scenarios such as texting, watching video, playing games, in-call progress. In another example, the label with clamping the UE 202 may represent that the UE 202 may be non-active and the user 204 may not be currently operating the UE 202. It may include scenario such as screen-off, UE 202 being inactive or unused by the user 204. Thus, the training dataset prepared classifies various scenario with the labels of either holding or clamping. The inertial sensor data during the clamping of the UE 202 may be less because of less free movement and similarly, the touch screen panel data may also be less thus indicating that the UE 202 is in a non-active or the user is clamping the UE 202.

At step 408, the method 400 may include the neural network being trained using the training dataset. In one or more embodiments, as the training dataset is fed to the neural network, the neural network may be adapted provide separation between the labels. The first vector feature is provided to the neural network as an input to predict the holding orientation of the UE 202. In an example, the holding orientation of the UE 202 may be that the user 204 is holding and currently operating the UE 202. In one or more embodiments, the neural network may be a sequential artificial neural network with fully connected layer and adapted to receive the first vector feature as input and provide two output possibility. In an example, an output layer of the neural network may provide the holding orientation of UE 202 as two probabilities, such as:
Y1: Probability indicating that the user 204 is holding and currently operating the UE 202;
Y2: Probability indicating that the user 204 is clamping the UE 202 and the UE 202 is not-in use.

In continuation with the step 408, at step 410, the method 400 may include the trained neural network is adapted to predict if the user 204 is holding and currently operating the UE 202. In one or more embodiments, the neural network is adapted to continuously predict, in real-time, the holding orientation of the UE 202 based on the inertial sensor data, the application type, and the touch screen panel data.

At step 412, the method 400 may include determining the body posture of the user 204 and the impacted body part upon finding the probability of the user 204 is holding and currently operating the UE 202.

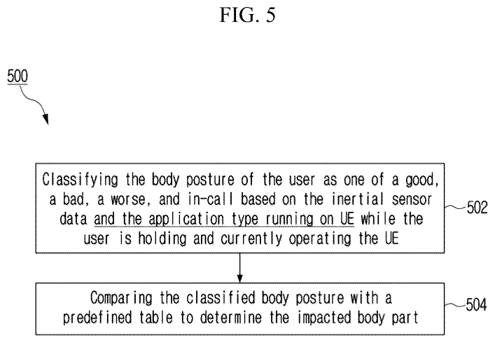

FIG. 5 illustrate an exemplary process flow comprising a method 500 for determining the body posture, according to one or more embodiments of the disclosure.

At step 510, the method 500 may include the neural network adapted to classify the body posture of the user 204. In an example, the body posture of the user 204 may be classified as one of a good, a bad, a worse, and in-call. In the method 500, the body posture is classified only if the neural network predicts that the user 204 is holding and currently operating the UE 202. In one or more embodiments, the body posture is classified based on the inertial sensor data and the application type running on UE 202. As depicted in FIG. 4A, the second vector feature is derived from the inertial sensor data. In an example, the inertial sensor data from the accelerometer, the gyroscope may provide an average angle. The average angel may be derived from an angle of usage of the UE 202, a duration of usage of the UE 202, a proximity of the UE 202 to face of the user 204. The average angle may form the input for the trained neural network. The trained neural network then determines the posture class as one of a good, a bad, a worse, and in-call based on the second vector feature provided as input.

At step 504, the method 500 may include comparing the classified body posture with a predefined table to determine the impacted body part. In one or more embodiments, the predefined table provides the impacted body part(s) corresponding to the classification of the body posture.

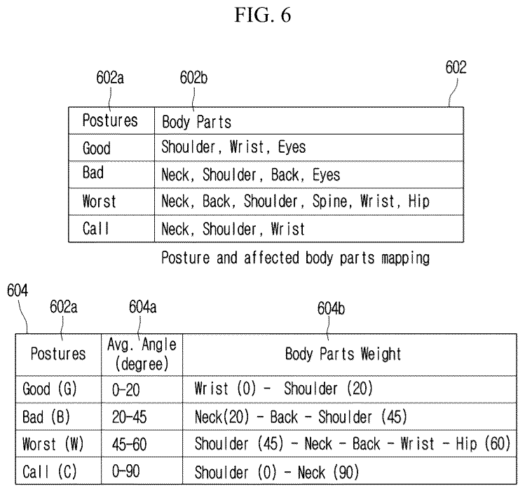

FIG. 6 illustrate an exemplary tables for determining the impacted body part, according to one or more embodiments of the disclosure.

The predefined table 602 depicts classification of the body posture 602a and corresponding impacted body part(s) 602b, mapped in the predefined table 602. In an example, for classification of the body posture 602a being good, the impacted body part(s) 602b of the user 204 may be a shoulder, a wrist, eyes. In an example, the classified body posture may impact more than body part as several of the body parts are interconnected as joints. For instance, as the body posture is classified as worst, the impacted body parts may be neck, back, shoulders such that neck and back are interconnected joints in the body.

Furthermore, the table 604 depicts a posture record including the classification of the body posture 602a corresponding to the average angle 604a. In an example, the average angle 604a is derived from the angle of usage of the UE 202, the duration of usage of the UE 202 and the proximity of the UE 202 to face of the user 204 as part of the inertial sensor data. At 604b in the table 604, is depicted the impacted body parts corresponding to the classification of the body posture 602a. In one or more embodiments, weights are assigned to each of the impacted body part for determining the impact level of each of the impacted body part. In an example, the posture record stores the average duration, average angle and affected body part data for further processing.

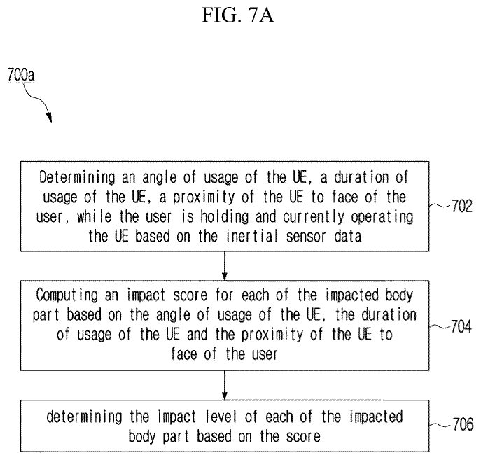

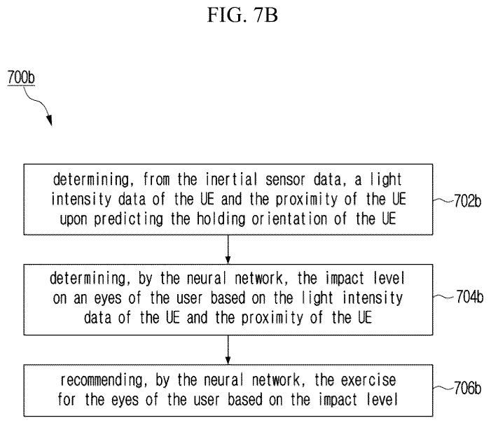

FIG. 7 illustrate an exemplary process flow comprising a method 700 for determining the impact level of the impacted body part, according to one or more embodiments of the disclosure.

At step 702, the method 700 may include determining the angle of usage of the UE 202, the duration of usage of the UE 202, the proximity of the UE 202 to face of the user 204, based on the inertial sensor data. In one or more embodiments, the impact level is calculated when the user 204 is holding and currently operating the UE 202. In an example, a score is calculated from the angle of usage of the UE 202 may be calculated using the formula:

Score(angle)=Σ_{Neck}^{Good,Bad,Worst,Call}weights per angle

In another example, a score is calculated from the duration of usage of the UE 202 may be calculated using the formula:

Score(duration)=Σ_{Neck}^{Good,Bad,Worst,Call}weights per duration

In an example, as the classified body posture at different N-second are determined, the neural network is adapted to derive an accumulated score by summation of the score (angle) and the score (duration).

In one or more embodiments, the weights per angle and weights per duration are predefined in the neural network.

Now, at step 704, the method 700 may include computing an impact score for each of the impacted body part based on the angle of usage of the UE, the duration of usage of the UE and the proximity of the UE to face of the user. In an example, the impact score may be computed based on deriving an occurrence of the impacted body part among the total number of impacted body parts. Further, the impact score is based on an average score calculated from the angle of usage and the duration of usage of the UE 202 respectively.

At step 706, the method 700 may include determining the impact level of each of the impacted body part based on the calculated impact score. In one or more embodiments, the impact level is one of a high level, a medium level, and a low level. The impact level is determined from the sum of maximum weights calculated. The threshold of calculated impact score using the above formula classifies the impact level to be one the high level, the medium level, or the low level.

In one or more embodiments, the method 700 may include determining the exercise repetition distribution based on the impact level. The exercise repetition distribution includes the frequency and the type of the exercise to be performed by the user 204. In an example, the type of the exercise is displayed based on the impact level determined.

In an example, the frequency in the exercise repetition distribution is calculated by the neural network. The neural network may calculate the exercise repetition distribution for the impacted body part based on the impact score, the type of exercise count and an occurrence count of the impact body part. In the example, the occurrence count indicates a normalized ratio of number of times a specific impacted body part has been recorded to be impacted over duration, out of total body parts appear in a posture record table.

Further, based on the determined exercise repetition distribution, the neural network derives the videos of the recommended type of exercise based on the at least one impacted body part and the impact level. In an example, the videos may be displayed on the UE 202 along with the determined exercise repetition distribution.

FIG. 7B illustrate an exemplary process flow comprising a method 700b for determining the impact level of the eyes of the user 204.

At step 702b, the method 700b may include determining a light intensity data of the UE 202 and the proximity of the UE 202 from face of the user 204 upon predicting that the user 204 is holding and currently operating the UE 202. In an example, the inertial sensor data may provide the a light intensity data of the UE 202 and the proximity of the UE 202 from face of the user 204.

At step 704b the method 700b may include determining by the neural network the impact level on the eyes of the user 204 based on the light intensity data of the UE 202 and the proximity of the UE 202.

At step 706b the method 700b may include recommending by the neural network, the exercise for the eyes of the user 203 based on the impact level determined.

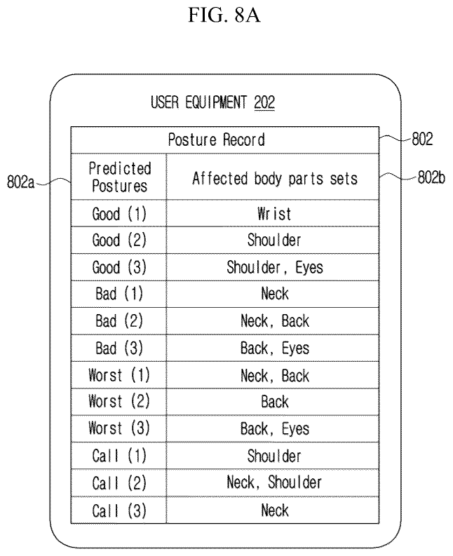

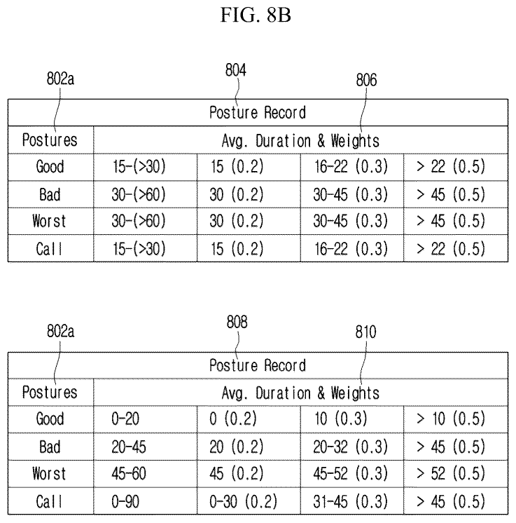

FIG. 8A illustrates an exemplary use case depicting the posture record table 802 providing the determined body posture 802a and the corresponding impacted body part 802b, according to one or more embodiments of the disclosure. As depicted in the FIG. 8A, the posture record table 802 may be saved in a database and is available for display on the UE 202.

FIG. 8B illustrates an exemplary use case depicting a predefined posture record table 804 providing the predefined postures 802a and corresponding an average duration and weights 806 for each of the predefined postures 802a. Another predefined posture record table 808 providing the predefined postures 802a and corresponding an average angle and weights 806 for each of the predefined postures 802a. The weights may be assigned based on research data and are provided to the neural network for calculating the impact score. In the present case use case, the impact sore is calculated for the body part-Neck:

Score(duration)=Σ_{Neck}^{Good,Bad,Worst,Call}weights per duration

Score(angle)=Σ_{Neck}^{Good,Bad,Worst,Call}weights per angle

Now, based on obtaining weights per duration and weights per angle from the predefined posture record table 804 and the predefined posture record table 808 respectively. In the example, the posture record table 804 depicts the body posture of the user 204 as bad for average duration of 30 minutes and assigned weight (0.2). Similarly, referring to the predefined posture record table 808 average angle, say 20 degrees and assigned weights (0.2). Therefore, the impact score calculated using the disclosure for the body part-Neck is as follows:

$$Score ( duration ) = Neck ( Call ( 0.3 ) + Worst ( 0.3 ) + Bad ( 0.2 ) ) = 0.8 \\ Score ( angle ) = Neck ( Call ( 0.2 ) + Worst ( 0.2 ) + Bad ( 0.2 ) ) = 0.6 \\ Average Score ( angle + duration ) = Neck ( ( 0.8 + 0.6 ) / 2 ) = 0.7$$

Thus, the impact score calculated for the impacted body part i.e., Neck is 0.70. Further, the calculated impact score classifies the impact level to be one the high level, the medium level, or the low level. A predefined threshold categorizing the impact level across a range of impact score may be used to classify the impact level. In the present exemplary example, the impact score may be classified as high level of impact level of the impacted body part-Neck. In the example, based on predefined table, a type of exercises is defined for each of the impact level. For example, for Neck two types of exercise may be provided i.e., rotation and stretching.

In the example, further the exercise repetition distribution for the neck is calculated. The exercise repetition distribution is based on the occurrence count, say it is equal to 3, the impact score, which is 0.70, type of exercise count is 2. Thus the exercise repetition distribution may be equal to approximately 3.

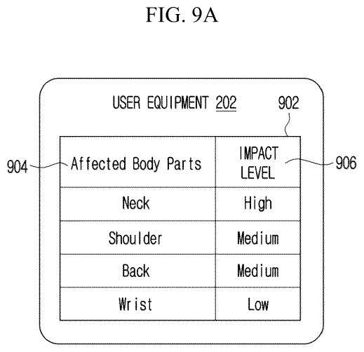

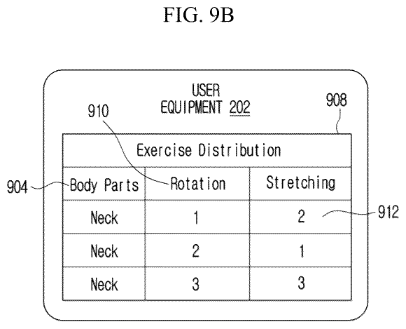

FIG. 9A illustrates another exemplary use case depicting a table 902 being displayed on the UE 202. The table 902 may include the impacted body part 904 and the impact level 906, according to one or more embodiments of the disclosure.

FIG. 9B illustrates another exemplary use case depicting a table 908 being displayed on the UE 202. The table 902 may include the impacted body part 904, the type of exercise 910, and the frequency 912 for the exercise according to one or more embodiments of the disclosure.

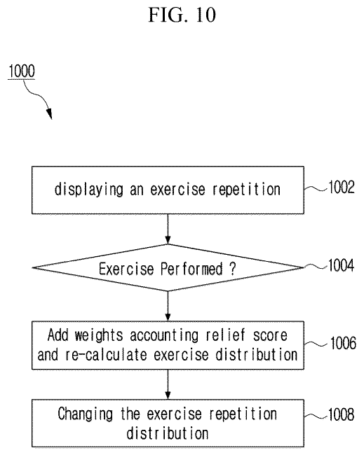

FIG. 10 illustrates an exemplary process flow comprising a method 1000 for receiving feedback from the user 204 and modifying the recommended exercises, according to one or more embodiments of the disclosure.

At step 1002, the method 1000 may include displaying the exercise repetition distribution as discussed in FIG. 3B.

At step 1004, the method 1000 may include receiving the feedback from the user 204 based on if the user 204 has performed the exercise. In one or more embodiments, the UE 202 may display a prompt message wherein the user 204 objectively provide the feedback indicating whether the user 204 performed the recommended exercise or not. Further, the user 204 may objectively provide the level of relief indicating the impact after performing the recommended exercise.

At step 1006, the method 1000 may include adjusting weights and re-calculating the exercise repetition distribution. In one or more embodiments, the re-calculation of the exercise repetition distribution may be computed by the neural network for the impacted body part based on the impact score, the type of exercise count and the occurrence count of the impact body part.

At step 1008, the method 1000 may include changing the exercise repetition distribution based on the feedback received from the user 204. In one or more embodiments the recommended videos may also be changed based on the changed exercise repetition distribution.

FIG. 11 illustrates a representative architecture 1100 to provide tools and implementation environment described herein for a technical realization of a system 1204 for determining impact on the body part while using the UE 202 and recommending the exercise. FIG. 11 is merely a non-limiting example, and it will be appreciated that many other architectures may be implemented to facilitate the functionality described herein. The architecture 1100 may be executing on hardware such as the UE 202 of FIG. 11 that includes, among other things, processors, memory, and various application-specific hardware components.

In the system 1104, the UE 202 may include an operating-system, libraries, frameworks or middleware. The operating system may manage hardware resources and provide common services. The operating system may include, for example, a kernel, services, and drivers defining a hardware interface layer. The drivers may be responsible for controlling or interfacing with the underlying hardware. For instance, the drivers may include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

A hardware interface layer includes libraries which may include system libraries such as filesystem (e.g., C standard library) that may provide functions such as memory allocation functions, string manipulation functions, mathematic functions, and the like. In addition, the libraries may include API libraries such as audio-visual media libraries (e.g., multimedia data libraries to support presentation and manipulation of various media format such as MPEG4, H.264, MP3, AAC, AMR, JPG, PNG), database libraries (e.g., SQLite that may provide various relational database functions), web libraries (e.g., WebKit that may provide web browsing functionality), and the like.

A middleware may provide a higher-level common infrastructure such as various graphic user interface (GUI) functions, high-level resource management, high-level location services, and so forth. The middleware may provide a broad spectrum of other APIs that may be utilized by the applications or other software components/modules, some of which may be specific to a particular operating system or platform.

The term “module” used in this disclosure may refer to a certain unit that includes one of hardware, software and firmware or any combination thereof. The module may be interchangeably used with unit, logic, logical block, component, or circuit, for example. The module may be the minimum unit, or part thereof, which performs one or more particular functions. The module may be formed mechanically or electronically. For example, the module disclosed herein may include at least one of ASIC (Application-Specific Integrated Circuit) chip, FPGAs (Field-Programmable Gate Arrays), and programmable-logic device, which have been known or are to be developed.

Further, the system 1204 in accordance with one or more embodiments of the present disclosure may include the UE 202 and the user 204. The UE 202 may include a set of instructions that can be executed via at least one processor 1112 to cause the UE 202 to perform any one or more of the methods disclosed. The UE 202 may operate as a standalone device or may be connected, e.g., using a network, to other computer systems or peripheral devices.

In one or more embodiments, the processor 1112 is configured to receive the inertial sensor data 1106 and the touch screen panel data 1108 of the UE 202. In an example the inertial sensor data 1106 is received from the accelerometer and the gyroscope installed in the UE 202. The processor 1112 is further configured to determine the application type running on the UE 202.

The processor 1112 is in communication with the neural network 1114 and is configured to predict, by the neural network 1114, the holding orientation of the UE 202 based on the inertial sensor data, the application type, and the touch screen panel data. In an example, the holding orientation of the UE 202 indicates whether the user 204 is holding and currently operating the UE 202.

In one or more embodiments, the processor 1112 is configured to determine, by the neural network 1114, the body posture of the user 204 and the impacted body part based on the inertial sensor data, in response to predicting that the user 204 is holding and currently operating the UE 202.

In one or more embodiments, the processor 1112 is configured to determine, by the neural network 1114, the impact level of the impacted body part based on the body posture, the holding orientation of the UE 202 and the inertial sensor data of the UE 202.

In one or more embodiments, the processor 1112 is configured to recommend the body posture correction and the exercise for the impacted body part based on the impact level.

FIG. 12 illustrates another system architecture of the system 1104 in the form of a computer system 1200. The computer system 1200 can include a set of instructions that can be executed to cause the computer system 1200 to perform any one or more of the methods disclosed. The computer system 1200 may operate as a standalone device or may be connected, e.g., using a network, to other computer systems or peripheral devices.

In a networked deployment, the computer system 1200 may operate in the capacity of a server or as a client user computer in a server-client user network environment, or as a peer computer system in a peer-to-peer (or distributed) network environment. The computer system 1200 can also be implemented as or incorporated across various devices, such as a personal computer (PC), a tablet PC, a personal digital assistant (PDA), a mobile device, a palmtop computer, a laptop computer, a desktop computer, a communications device, a wireless telephone, a land-line telephone, a web appliance, a network router, switch or bridge, or any other machine capable of executing a set of instructions (sequential or otherwise) that specify actions to be taken by that machine. Further, while a single computer system 1200 is illustrated, the term “system” shall also be taken to include any collection of systems or sub-systems that individually or jointly execute a set, or multiple sets, of instructions to perform one or more computer functions.

The computer system 1200 may include the processor 1112 e.g., a central processing unit (CPU), a graphics processing unit (GPU), or both. The processor 1112 may be a component in a variety of systems. For example, the processor 1112 may be part of a standard personal computer or a workstation. The processor 1112 may be one or more general processors, digital signal processors, application-specific integrated circuits, field-programmable gate arrays, servers, networks, digital circuits, analog circuits, combinations thereof, or other now known or later developed devices for analyzing and processing data. The processor 1112 may implement a software program, such as code generated manually (i.e., programmed).

The computer system 1200 may include a memory 1208, such as a memory 1208 that can communicate via a bus 1218. The memory 1208 may include but is not limited to computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one example, memory 1208 includes a cache or random-access memory for the processor 1112. In alternative examples, the memory 1208 is separate from the processor 1112, such as a cache memory of a processor, the system memory, or other memory. The memory 1208 may be an external storage device or database for storing data. The memory 1208 is operable to store instructions executable by the processor 1112. The functions, acts or tasks illustrated in the figures or described may be performed by the programmed processor 1112 for executing the instructions stored in the memory 1208. The functions, acts or tasks are independent of the particular type of instructions set, storage media, processor or processing strategy and may be performed by software, hardware, integrated circuits, firmware, micro-code and the like, operating alone or in combination. Likewise, processing strategies may include multiprocessing, multitasking, parallel processing and the like.

As shown, the computer system 1200 may or may not further include a display 1210, such as a liquid crystal display (LCD), an organic light-emitting diode (OLED), a flat panel display, a solid-state display, a cathode ray tube (CRT), a projector, a printer or other now known or later developed display device for outputting determined information. The display 1210 may act as an interface for the user to see the functioning of the processor 1112, or specifically as an interface with the software stored in the memory 1208 or the drive unit 1216.

Additionally, the computer system 1200 may include an input device 1212 configured to allow the user to interact with any of the components of system 1204. The computer system 1200 may also include a disk or optical drive unit 1216. The disk drive unit 1216 may include a computer-readable medium 1222 in which one or more sets of instructions 1224, e.g., software, can be embedded. Further, the instructions 1224 may embody one or more of the methods or logic as described. In a particular example, the instructions 1224 may reside completely, or at least partially, within the memory 1208 or within the processor 1112 during execution by the computer system 1200.

The disclosure contemplates a computer-readable medium that includes instructions 1224 or receives and executes instructions 1224 responsive to a propagated signal so that a device connected to a network 1226 can communicate voice, video, audio, images, or any other data over the network 1226. Further, the instructions 1224 may be transmitted or received over the network 1226 via a communication port or interface 1220 or using a bus 1218. The communication port or interface 1220 may be a part of the processor 1206 or maybe a separate component. The communication port 1220 may be created in software or maybe a physical connection in hardware. The communication port 1220 may be configured to connect with a network 1226, external media, the display 1210, or any other components in system 1204, or combinations thereof. The connection with the network 1226 may be a physical connection, such as a wired Ethernet connection or may be established wirelessly as discussed later. Likewise, the additional connections with other components of the system 1204 may be physical or may be established wirelessly. The network 1226 may alternatively be directly connected to the bus 1218.

The network 1226 may include wired networks, wireless networks, Ethernet AVB networks, or combinations thereof. The wireless network may be a cellular telephone network, an 802.11, 802.16, 802.20, 802.1Q or WiMax network. Further, the network 826 may be a public network, such as the Internet, a private network, such as an intranet, or combinations thereof, and may utilize a variety of networking protocols now available or later developed including, but not limited to TCP/IP based networking protocols. The system is not limited to operation with any particular standards and protocols. For example, standards for Internet and other packet-switched network transmissions (e.g., TCP/IP, UDP/IP, HTML, and HTTP) may be used.

While specific language has been used to describe the disclosure, any limitations arising on account of the same are not intended. As would be apparent to a person in the art, various working modifications may be made to the method in order to implement the inventive concept as taught herein.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein.

What is claimed is:

1. A method for determining impact on at least one body part while using a user equipment (UE) and recommending at least one exercise, the method comprising:
receiving inertial sensor data and touch screen data of the user equipment (UE);
determining an application type running on the UE;
predicting, by a neural network, a holding orientation of the UE based on the inertial sensor data, the application type, and the touch screen data, wherein the holding orientation indicates whether a user is holding and currently operating the UE;
determining, by the neural network, a body posture of the user and at least one impacted body part based on the inertial sensor data, based on the holding orientation;
determining, by the neural network, an impact level of the at least one impacted body part based on the body posture, the holding orientation and the inertial sensor data; and
recommending a body posture correction and the at least one exercise for the at least one impacted body part based on the impact level.

2. The method as claimed in claim 1, wherein the recommending the body posture correction and the at least one exercise comprises:
displaying an exercise repetition distribution indicating a frequency and a type of the at least one exercise to be performed by the user; and
displaying a video for the at least one exercise based on the at least one impacted body part and the impact level.

3. The method as claimed in claim 2, further comprising:
receiving, by a touch screen of the UE, feedback from the user while the user performs the at least one exercise for the at least one impacted body part; and
updating the exercise repetition distribution based on the feedback.

4. The method as claimed in claim 1, wherein the determining the body posture of the user and the at least one impacted body part comprises:
classifying the body posture of the user as one of good, bad, worse, or in-call based on the inertial sensor data and the application type running on UE while the user is holding and currently operating the UE; and
comparing the classified body posture with a predefined table to determine the at least one impacted body part, wherein the predefined table indicates the at least one body part corresponding to the classifying of the body posture.

5. The method as claimed in claim 1, wherein the determining the impact level of the at least one impacted body part based on the body posture comprises:
determining an angle of usage of the UE, a duration of usage of the UE, a proximity of the UE to a face of the user, while the user is holding and currently operating the UE based on the inertial sensor data;
computing an impact score for each of the at least one impacted body part based on the angle of usage, the duration of usage and the proximity to the face of the user; and
determining the impact level of each of the at least one impacted body part based on the impact score, wherein the impact level is indicated as one of a high level, a medium level, and a low level.

6. The method as claimed in claim 5, further comprising:
determining, from the inertial sensor data, light intensity data of the UE and the proximity upon predicting the holding orientation;
determining, by the neural network, the impact level on eyes of the user based on the light intensity data and the proximity; and
recommending, by the neural network, the at least one exercise for the eyes of the user based on the impact level.

7. The method as claimed in claim 1, wherein the inertial sensor data comprises data from at least one of an accelerometer and a gyroscope.

8. The method as claimed in claim 1, wherein the touch screen data comprises at least one of touch coordinates, a hover distribution, and a duration of touch on a touch screen of the UE.

9. A system for determining impact on at least one body part while using a user equipment (UE) and recommending at least one exercise, the system comprising:
memory storing instructions; and
at least one processor configured to execute the instructions,
wherein the instructions, when executed by the at least one processor, cause the system to:
receive inertial sensor data and touch screen data of the UE;
determine an application type running on the UE;
predict, by a neural network, a holding orientation of the UE based on the inertial sensor data, the touch screen data, and the application type, wherein the holding orientation of the UE indicates whether a user is holding and currently operating the UE;
determine, by the neural network, a body posture of the user and at least one impacted body part based on the inertial sensor data, based on the holding orientation;
determine, by the neural network, an impact level of the at least one impacted body part based on the body posture, the holding orientation and the inertial sensor data; and
recommend a body posture correction and the at least one exercise for the at least one impacted body part based on the impact level.

10. The system as claimed in claim 9, wherein the instructions, when executed by the at least one processor, cause the system to:
control a touch screen of the UE to display an exercise repetition distribution indicating a frequency and a type of the at least one exercise to be performed by the user; and
control the touch screen to display a video for the at least one exercise based on the at least one impacted body part and the impact level.

11. The system as claimed in claim 10, the instructions, when executed by the at least one processor, cause the system to:
receive, by the touch screen, feedback from the user while the user performs the at least one exercise for the at least one impacted body part; and
update the exercise repetition distribution based on the feedback.

12. The system as claimed in claim 9, wherein the instructions, when executed by the at least one processor, cause the system to:
classify the body posture of the user as one of good, bad, worse, or in-call based on the inertial sensor data and the application type running on UE while the user is holding and currently operating the UE; and
compare the classified body posture with a predefined table to determine the at least one impacted body part, wherein the predefined table indicates the at least one body part corresponding to the classifying of the body posture.

13. The system as claimed in claim 9, wherein the instructions, when executed by the at least one processor, cause the system to:
determine an angle of usage of the UE, a duration of usage of the UE, a proximity of the UE to a face of the user, while the user is holding and currently operating the UE based on the inertial sensor data;
compute an impact score for each of the at least one impacted body part based on the angle of usage, the duration of usage and the proximity to the face of the user; and
determine the impact level of each of the at least one impacted body part based on the impact score, wherein the impact level is indicated as one of a high level, a medium level, and a low level.

14. The system as claimed in claim 13, the instructions, when executed by the at least one processor, cause the system to:
determine, from the inertial sensor data, a light intensity data of the UE and the proximity upon predicting the holding orientation;
determine, by the neural network, the impact level on eyes of the user based on the light intensity data and the proximity; and
recommend, by the neural network, the at least one exercise for the eyes of the user based on the impact level.

15. The system as claimed in claim 9, wherein the inertial sensor data comprises data from at least one of an accelerometer and a gyroscope.

16. A user equipment (UE) comprising:
an inertial sensor;
a touch screen;
memory storing instructions; and
at least one processor configured to executed the instructions,
wherein the instructions, the instructions, when executed by the at least one processor, cause the system:
receive inertial sensor data from the inertial sensor and touch screen data from the touch screen;
predict, by a neural network, a holding orientation of the UE based on the inertial sensor data and the touch screen data;
determine, by the neural network, a body posture of a user and a first body part and a second body part impacted by the body posture, based on the holding orientation;
determine, by the neural network, a first impact level of the first body part and a second impact level of the second body part based on the body posture; and
recommend a body posture correction and at least one exercise for at least one of the first body part or the second body part, based on the first impact level and the second impact level.
